(19) United States
(12) Patent Application Publication
(10) Pub. No.: US 2015/0080721 A1
(43) Pub. Date: Mar. 19, 2015

(54) DETECTION OF TUMOR MICROENVIRONMENT WITH CHLOROTOXIN CONJUGATES

(71) Applicants: Blaze Bioscience, Inc., Seattle, WA (US); Washington State University, Pullman, WA (US)

(72) Inventors: Julia E. Novak, Suquamish, WA (US); Stacey J. Hansen, Renton, WA (US); William S. Dernell, Viola, ID (US); Katie C. Kennedy, Pullman, WA (US); Valorie R. Wiss, Moscow, ID (US)

(21) Appl. No.: 14/489,419

(22) Filed: Sep. 17, 2014

Related U.S. Application Data
(60) Provisional application No. 61/879,096, filed on Sep. 17, 2013.
(60) Provisional application No. 61/990,101, filed on May 7, 2014.

(51) Int. Cl. A61K 49/00; A61B 5/00; G01N 33/574

(52) U.S. Cl. CPC A61K 49/0056; G01N 33/5743; G01N 33/57415; G01N 33/57407; A61B 5/0071; A61B 5/0075; G01N 2458/00
USPC 600/431; 424/9.6; 435/29; 530/324

(57) ABSTRACT

The present disclosure provides methods for detecting a tumor microenvironment, a peritumoral tissue, or a portion thereof using a chlorotoxin conjugate. Also provided are chlorotoxin peptides and variants thereof conjugated to a detectable label for use in detecting a tumor microenvironment, a peritumoral tissue, or a portion thereof.

### CROSS-REFERENCE

This application claims the benefit of U.S. Provisional Application No. 61/879,096, filed Sep. 17, 2013 and U.S. Provisional Application No. 61/990,101, filed May 7, 2014, which are incorporated herein by reference in their entirety.

### STATEMENT AS TO FEDERALLY SPONSORED RESEARCH

This invention was made with the support of the United States government by the National Cancer Institute, National Institutes of Health, Department of Health and Human Services, under Contract No. HHSN261201200054C.

### BACKGROUND OF THE INVENTION

Tumor microenvironment is the cellular environment in which a tumor exists, including surrounding blood vessels, immune cells, fibroblasts, other cells, signaling molecules, and the extracellular matrix (ECM). The tumor and the surrounding microenvironment are closely related and interact constantly. Improved methods for detection of tumor microenvironment are needed to improve a surgeon's ability to detect and possibly remove the tumor microenvironment and/or peritumoral tissue that may become cancerous in certain cases, such as cutaneous squamous cell carcinoma and breast or mammary cancer. The present invention provides such methods of detection, imaging, visualization and analysis for these and other uses that should be apparent to those skilled in the art from the teachings herein.

### SUMMARY OF THE INVENTION

In various aspects, the present disclosure provides methods of detecting a tumor microenvironment, peritumoral tissue, or cells therefrom, the method comprising the steps of: administering a chlorotoxin conjugate to the tumor microenvironment, peritumoral tissue, or cells therefrom, wherein the chlorotoxin conjugate binds to the tumor microenvironment, peritumoral tissue, cells therefrom, or a combination thereof; and imaging, visualizing, or analyzing the bound chlorotoxin conjugate.

of detecting a tumor microenvironment or cells therefrom, the method comprising the steps of: administering a chlorotoxin conjugate to the tumor microenvironment or cells therefrom, wherein the chlorotoxin conjugate binds to the tumor microenvironment or cells therefrom; and imaging, visualizing, or analyzing the bound chlorotoxin conjugate.

In some aspects of the present disclosure, the methods further comprise removing the tumor microenvironment or cells therefrom bound by the chlorotoxin conjugate.

In certain aspects of the present disclosure, the tumor microenvironment or cells therefrom are associated with tumors of skin or breast.

In additional aspects of the present disclosure, the chlorotoxin conjugate is administered to an individual.

In some aspects of the present disclosure, the imaging, visualizing, or analyzing comprises visualizing the chlorotoxin conjugate optically.

In further aspects of the present disclosure, the imaging, visualizing, or analyzing comprises in vivo or ex vivo imaging, visualizing, or analyzing.

In certain aspects of the present disclosure, the imaging, visualizing, or analyzing comprises optically imaging the tumor microenvironment.

In some aspects of the present disclosure, the chlorotoxin conjugate comprises a detectable label.

In further aspects of the present disclosure, the detectable label comprises a fluorescent moiety.

In additional aspects of the present disclosure, the fluorescent moiety comprises a near infrared fluorescent moiety.

In certain aspects of the present disclosure, the detectable label comprises a cyanine dye.

In some aspects of the present disclosure, the detectable label is selected from the group consisting of Cy5.5, DyLight 750, indocyanine green (ICG) and IRdye 800.

In additional aspects of the present disclosure, the detectable label comprises a radionuclide.

In further aspects of the present disclosure, the detecting is performed during or related to surgery or resection.

In certain aspects of the present disclosure, the chlorotoxin comprises a sequence having at least 85% sequence identity to the sequence of MCMPCFTTDHQMARXCDDCCGGXGRGXCYGPQCLCR, wherein X is selected from K, A and R.

In further aspects of the present disclosure, the imaging, visualizing, or analyzing the bound chlorotoxin conjugate is performed on a sample.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows intraoperative imaging of a canine cutaneous squamous cell carcinoma. (A) White light preoperative image of tumor site, showing grossly ulcerated and swollen peritumoral skin. Panels B and C show preoperative NIR fluorescence images of the tumor from the top (B) and side (C). NIR fluorescence (D) and overlay (E) are shown following removal of the tail. The mass at right is a section of central tumor removed for further analysis. The skin is retracted, and the remaining gross tumor (arrow) is revealed. Fluorescence intensity is similar in the central tumor and remaining gross tumor, while peritumoral skin has somewhat lower fluorescence intensity (F). Tissues were labeled with a chlorotoxin-dye conjugate.

FIG. 2 shows a grid analysis for sensitivity and specificity. (A) Fluorescence image with overlaid squares (2 mm×2 mm). Total fluorescence in each square was measured. (B) H&E stain with tumor outlined. Tissues were labeled with a chlorotoxin-dye conjugate.

FIG. 3 shows a box & whiskers plots of fluorescence intensity in grid squares for each tissue section analyzed. T, tumor. NT, adjacent non-tumor tissue. PS, peritumoral skin. S, uninvolved skin. For the cutaneous tumors, the NT consisted of underlying dermis, subcutaneous fat, and adjacent dermis/epidermis. Tissues were labeled with a chlorotoxin-dye conjugate.

### DETAILED DESCRIPTION

The inventors have identified that chlorotoxin conjugates can be used to detect and identify tumor microenvironments or peritumoral tissue that may be likely to become tumor tissue in certain tissues, such as cutaneous squamous cell carcinoma and breast or mammary cancer.

The term “tumor microenvironment” as used herein refers to the non-cancerous cells, molecules, and blood vessels that surround and potentially feed a tumor cell. A tumor can change its microenvironment, and the microenvironment can affect how a tumor grows and spreads. Identification of tumor microenvironment in intraoperative tumor resection can be useful as a means of identifying if the tissues of the original site may become cancerous in the future, especially in breast and skin tissues.

The present disclosure also provides methods of using a chlorotoxin conjugate to inhibit, prevent, minimize, shrink, or kill cells, or prevent metastasis in a tumor microenvironment. In one aspect, the tumor microenvironment is from a cutaneous squamous cell carcinoma tumor. In another aspect, the tumor microenvironment is from a mammary carcinoma tumor.

In another aspect a method is provided for inhibiting, preventing, minimizing, shrinking, or killing cells, or preventing metastasis in a tumor microenvironment in an individual, comprising the step of administering a chlorotoxin conjugate to the individual wherein the chlorotoxin conjugate binds to the tumor microenvironment or cells; and whereby peritumoral cells in the tumor microenvironment are inhibited, prevented, minimized, shrunk, or killed, or metastasis is prevented. In one embodiment, the chlorotoxin conjugate comprises a chemotherapeutic, a radionuclide, an anti-cancer agent, or an anti-cancer drug. In another embodiment, the chlorotoxin conjugate comprising the chemotherapeutic, an anti-cancer agent, or an anti-cancer drug is administered after the central or primary tumor is detected during surgery. In a further embodiment, the central or primary tumor is detected with a chlorotoxin conjugated to a labeling agent.

In another aspect the invention provides, a method of administering a chlorotoxin conjugated to a chemotherapeutic, an anti-cancer agent, or an anti-cancer drug to an individual to treat, inhibit, prevent, minimize, shrink, or kill cells, or prevent metastasis in cells that are identified with a chlorotoxin conjugated to a labeling agent, wherein the cells are not tumor cells. In one embodiment, the cells are peritumoral cells. In another embodiment, the cells are in the tumor microenvironment. In another embodiment, the cells are in the tumor bed. In some embodiments the anti-cancer agent includes antibodies, polypeptides, polysaccharides, or nucleic acids. In an embodiment, the chlorotoxin conjugate is administered about 1 day before the surgery. In another embodiment, the chlorotoxin conjugate is administered about 2 days before surgery. In another embodiment, the chlorotoxin conjugate is administered in multiple sub-doses. In an embodiment, the chlorotoxin conjugate is administered in about 2 sub-doses, 3 sub-doses, 4 sub-doses, or more sub-doses. In another embodiment, the chlorotoxin conjugate comprising the chemotherapeutic, an anti-cancer agent, or an anti-cancer drug is administered after the central or primary tumor is detected during surgery. In a further embodiment, the central or primary tumor is detected with a chlorotoxin conjugated to a labeling agent.

### Chlorotoxin Conjugates

In various aspects, chlorotoxin conjugates comprise a chlorotoxin peptide and a labeling agent or detectable label. In various aspects, chlorotoxin conjugates comprise a chlorotoxin peptide and a labeling agent or detectable label. In an embodiment, chlorotoxin is a variant comprising at least 60%, 65%, 70%, 75%, 80%, 83%, 85%, 86%, 89%, 90%, 92%, 93%, 94%, 95%, 96%, 97%, 98%, or 99% identical to the sequence of the natural peptide of chlorotoxin. In another embodiment, the present disclosure provides a chlorotoxin having the following amino acid sequence: MCMPCFTTDHQMARKCDDCCGGKGRGKCYGPQCLCR. In a further embodiment, the present disclosure provides chlorotoxin variants comprising at least 60%, 65%, 70%, 75%, 80%, 83%, 85%, 86%, 89%, 90%, 92%, 93%, 94%, 95%, 96%, 97%, 98%, or 99% identical to the following amino acid sequence: MCMPCFTTDHQMARKCDDCCGGKGRGKCYGPQCLCR. In another embodiment, the chlorotoxin is a chlorotoxin or variant thereof comprising at least 60%, 65%, 70%, 75%, 80%, 83%, 85%, 86%, 89%, 90%, 92%, 93%, 94%, 95%, 96%, 97%, 98%, or 99% sequence identity to the sequence of MCMPCFTTDHQMARXCDDCCGGXGRGXCYGPQCLCR, wherein X is selected from K, A and R. In another embodiment, the chlorotoxin is a chlorotoxin or variant of thereof comprising at least 85% sequence identity to the sequence of MCMPCFTTDHQMARXCDDCCGGXGRGXCYGPQCLCR, wherein X is selected from K, A and R. The peptide can be further cross-linked by four disulfide bonds formed among the cysteine residues present in the sequence. In some embodiments, the chlorotoxin can be a chlorotoxin variant. Chlorotoxin and chlorotoxin variants have are further described in PCT Patent Application Publication Numbers WO2006115633 and WO2011142858, which are incorporated in their entirety herein by reference.

In one embodiment, the peptide can have the following formula: H-Met-Cys-Met-Pro-Cys-Phe-Thr-Thr-Asp-His-Gln-Met-Ala-Arg-Xaa-Cys-Asp-Asp-Cys-Cys-Gly-Gly-Xaa-Gly-Arg-Gly-Xaa-Cys-Tyr-Gly-Pro-Gln-Cys-Leu-Cys-Arg-OH acetate salt (disulfide bonds, air oxidized), wherein Xaa is Arg, Ala, or Lys.

In another embodiment, the all peptide can have the following formula: H-Met-Cys-Met-Pro-Cys-Phe-Thr-Thr-Asp-His-Gln-Met-Ala-Arg-Xaa-Cys-Asp-Asp-Cys-Cys-Gly-Gly-Xaa-Gly-Arg-Gly-Lys-Cys-Tyr-Gly-Pro-Gln-Cys-Leu-Cys-Arg-OH acetate salt (disulfide bonds, air oxidized), wherein Xaa is Arg, or Ala.

In another embodiment, the peptide can have the following formula: H-Met-Cys-Met-Pro-Cys-Phe-Thr-Thr-Asp-His-Gln-Met-Ala-Arg-Arg-Cys-Asp-Asp-Cys-Cys-Gly-Gly-Arg-Gly-Arg-Gly-Lys-Cys-Tyr-Gly-Pro-Gln-Cys-Leu-Cys-Arg-OH acetate salt (disulfide bonds, air oxidized).

In another embodiment, the peptide can have the following formula: H-Met-Cys-Met-Pro-Cys-Phe-Thr-Thr-Asp-His-Gln-Met-Ala-Arg-Arg-Cys-Asp-Asp-Cys-Cys-Gly-Gly-Ala-Gly-Arg-Gly-Lys-Cys-Tyr-Gly-Pro-Gln-Cys-Leu-Cys-Arg-OH acetate salt (disulfide bonds, air oxidized).

In another embodiment, the peptide can have the following formula: H-Met-Cys-Met-Pro-Cys-Phe-Thr-Thr-Asp-His-Gln-Met-Ala-Arg-Ala-Cys-Asp-Asp-Cys-Cys-Gly-Gly-Arg-Gly-Arg-Gly-Lys-Cys-Tyr-Gly-Pro-Gln-Cys-Leu-Cys-Arg-OH acetate salt (disulfide bonds, air oxidized).

In another embodiment, the peptide can have the following formula: H-Met-Cys-Met-Pro-Cys-Phe-Thr-Thr-Asp-His-Gln-Met-Ala-Arg-Ala-Cys-Asp-Asp-Cys-Cys-Gly-Gly-Ala-Gly-Arg-Gly-Lys-Cys-Tyr-Gly-Pro-Gln-Cys-Leu-Cys-Arg-OH acetate salt (disulfide bonds, air oxidized).

In accordance, e.g., with the methods of making peptides of the present invention, a wide variety of variants of chlorotoxin can be generated. For example, using conventional mutagenesis and synthetic-based systems, thousands of non-natural amino acids can be prepared based on the naturally occurring amino acid sequence of chlorotoxin. In some embodiments, the peptides of the present invention can include an amino acid sequence at least 60%, 65%, 70%, 75%, 80%, 83%, 85%, 86%, 89%, 90%, 92% or 95% identical to the following sequence of MCMPCFTTDHQMARKCDDCCGGKGRGKCYGPQCLCR, in which some or all of the amino acids are non-natural amino acids. In some embodiments, the chlorotoxin variants can have at least three of the amino acids in the sequence that are a non-natural amino acid in place of the natural amino acid. In some embodiments, at least four, at least 5, at least 10, at least 15, at least 20, at least 25, at least 30, or at least 35 of the amino acids in the sequence of the chlorotoxin variants can include a non-natural amino acid in place of a natural amino acid. In certain embodiments, at least 10%, at least 20%, at least 30%, at least 40%, at least 50%, at least 60%, at least 70%, at least 80%, or at least 90% of the amino acids in the sequence of the chlorotoxin variants can include a non-natural amino acid in place of a natural amino acid.

In some aspects, chlorotoxin and chlorotoxin variants can be conjugated with a variety of moieties that can, e.g., modify the pharmacological properties of the peptides. In some embodiments, some or all of the lysines in the amino acid sequence of the peptides can be replaced with alanine or arginine to facilitate N-terminal conjugation (e.g., by reducing competition from the free amine of the lysine). In some embodiments, some or all, one, or two of the lysines in the amino acid sequence of the peptides can be blocked such that they are not available for conjugation.

For example, the present invention can include chlorotoxin and chlorotoxin variants that have an amino acid sequence at least 60%, 65%, 70%, 75%, 80%, 83%, 85%, 86%, 89%, 90%, 92% or 95% identical to the following sequence of MCMPCFTTDHQMARXCDDCCGGXGRGXCYGPQCLCR, in which some or all of the amino acids are non-natural amino acids and X can include K, A or R.

The chlorotoxin and chlorotoxin variant conjugates can include moieties that are conjugated to various locations of the peptides. For example, moieties can be conjugated to any one of the lysine residues in chlorotoxin sequence (e.g., Lys-15, Lys-23, and/or Lys-27). Alternatively, the peptides may be mutated to be free of lysine residues. In some embodiments, the moieties can be conjugated to the N-terminus of the chlorotoxin peptides and chlorotoxin peptide variants. In some embodiments, the moieties can be conjugated to at least one of the amino acids in the peptides.

In certain embodiments, the chlorotoxin and chlorotoxin variants can be conjugated to moieties, such as detectable labels (e.g., dyes) that can be detected (e.g., visualized) in a subject. In some embodiments, the chlorotoxin and/or chlorotoxin variants can be conjugated to detectable labels to enable tracking of the bio-distribution of a conjugated peptide. The detectable labels can include fluorescent labels (e.g., fluorescent dyes).

In certain embodiments, the chlorotoxin and chlorotoxin variants can be conjugated to moieties, such as detectable labels (e.g., dyes) that can be detected (e.g., visualized) in a subject. In some embodiments, the chlorotoxin and/or chlorotoxin variants can be conjugated to detectable labels to enable tracking of the bio-distribution of a conjugated peptide. The detectable labels can include fluorescent dyes. Non-limiting examples of fluorescent dyes that could be used as a conjugating molecule in the present disclosure include rhodamine, rhodol, fluorescein, thiofluorescein, aminofluorescein, carboxyfluorescein, chlorofluorescein, methylfluorescein, sulfofluorescein, aminorhodol, carboxyrhodol, chlororhodol, methylrhodol, sulforhodol; aminorhodamine, carboxyrhodamine, chlororhodamine, methylrhodamine, sulforhodamine, and thiorhodamine, cyanine, indocarbocyanine, oxacarbocyanine, thiacarbocyanine, merocyanine, a cyanine dye (e.g., cyanine 2, cyanine 3, cyanine 3.5, cyanine 5, cyanine 5.5, cyanine 7), oxadiazole derivatives, pyridyloxazole, nitrobenzoxadiazole, benzoxadiazole, pyrene derivatives, cascade blue, oxazine derivatives, Nile red, Nile blue, cresyl violet, oxazine 170, acridine derivatives, proflavin, acridine orange, acridine yellow, arylmethine derivatives, auramine, xanthene dyes, sulfonated xanthenes dyes, Alexa Fluors (e.g., Alexa Fluor 594, Alexa Fluor 633, Alexa Fluor 647, Alexa Fluor 700), crystal violet, malachite green, tetrapyrrole derivatives, porphyrin, phtalocyanine, and bilirubin. Some other example dyes include near-infrared dyes, such as, but not limited to, Cy5.5, indocyanine green (ICG), DyLight 750 or IRdye 800. In some embodiments, near infrared dyes can include cyanine dyes.

As used herein, the terms “about” and “approximately,” in reference to a number, is used herein to include numbers that fall within a range of 10%, 5%, or 1% in either direction (greater than or less than) the number unless otherwise stated or otherwise evident from the context (except where such number would exceed 100% of a possible value).

Suitable diagnostic agents include agents that provide for the detection by fluorescence methods as well as methods other than fluorescence imaging. Other suitable diagnostic agents include radiolabels (e.g., radio isotopically labeled compounds) such as ¹²⁵I, ¹⁴C, and ³¹P, among others; and magnetic resonance imaging agents.

Suitable targeting agents include antibodies, polypeptides, polysaccharides, and nucleic acids.

### Imaging Methods

In a further aspect of the invention, methods of using the chlorotoxin conjugates are provided. In one embodiment, the invention provides a method for imaging tumor microenvironment using a chlorotoxin conjugate. In the method, tumor microenvironment is contacted with a chlorotoxin conjugate. In one embodiment, the imaging method is a fluorescence imaging method. Representative methods for making and using fluorescent chlorotoxin conjugates are described in U.S. Patent Application Publication No. 20080279780 A1, Fluorescent Chlorotoxin Conjugate and Method for Intra-Operative Visualization of Cancer, and in U.S. Patent Application Publication No. 20130195760, Chlorotoxin Variants, Conjugates, And Methods For Their Use, both of which are expressly incorporated herein by reference in their entirety.

The present invention provides methods for intraoperative imaging and resection of tumor microenvironment with a chlorotoxin conjugate detectable by fluorescence imaging that allows for intraoperative visualization of tumor microenvironment. In one embodiment, the chlorotoxin conjugate of the invention includes one or more labeling agents. In a further embodiment, the labeling agent comprises a fluorescent moiety (e.g., red or near infrared emitting fluorescent moieties) covalently coupled to the chlorotoxin. In another embodiment, the labeling agent comprises a radionuclide.

As used herein, the term “red or near infrared emitting fluorescent moiety” refers to a fluorescent moiety having a fluorescence emission maximum greater than about 600 nm.

In certain embodiments of the chlorotoxin conjugate, the fluorescent moieties are derived from fluorescent compounds characterized by emission wavelength maxima greater than about 600 nm to avoid auto-fluorescence, emission that travels through millimeters to one centimeter of tissue/blood/fluids, emission that is not absorbed by hemoglobin, other blood components, or proteins in human or animal tissue.

The fluorescent moiety is covalently coupled to the chlorotoxin to allow for the visualization of the conjugate by fluorescence imaging. The fluorescent moiety is derived from a fluorescent compound. Suitable fluorescent compounds are those that can be covalently coupled to a chlorotoxin without substantially adversely affecting the targeting and binding function of the chlorotoxin conjugate. Similarly, suitable fluorescent compounds retain their fluorescent properties after conjugation to the chlorotoxin.

Generally, the dosage of administered chlorotoxin conjugates may vary depending upon such factors as the patient's age, weight, height, sex, general medical condition and previous medical history. Typically, it is desirable to provide the recipient with a dosage of a chlorotoxin conjugate that is in the range of from about 3 mg to about 6 mg, although a lower or higher dosage also may be administered as circumstances dictate.

Administration of a chlorotoxin conjugate to a subject can be topical, inhalant, intravenous, intraarterial, intraperitoneal, intramuscular, subcutaneous, intrapleural, intrathecal, by perfusion through a regional catheter, or by direct intralesional injection. When administering conjugates by injection, the administration may be by continuous infusion or by single or multiple boluses.

Additional routes of administration include oral, mucosal-membrane, pulmonary, and transcutaneous. Oral delivery is suitable for polyester microspheres, zein microspheres, proteinoid microspheres, polycyanoacrylate microspheres, and lipid-based systems (see, for example, DiBase and Morrel, “Oral Delivery of Microencapsulated Proteins,” in Protein Delivery: Physical Systems, Sanders and Hendren (eds.), pages 255-288 (Plenum Press 1997)). The feasibility of an intranasal delivery is exemplified by such a mode of insulin administration (see, for example, Hinchcliffe and Ilium, Adv. Drug Deliv. Rev. 35:199 (1999)). Dry or liquid particles comprising a chlorotoxin conjugate can be prepared and inhaled with the aid of dry-powder dispersers, liquid aerosol generators, or nebulizers (e.g., Pettit and Gombotz, TIBTECH 16:343 (1998); Patton et al., Adv. Drug Deliv. Rev. 35:235 (1999)). This approach is illustrated by the AERX diabetes management system, which is a hand-held electronic inhaler that delivers aerosolized insulin into the lungs. Transdermal delivery using electroporation provides another means to administer a chlorotoxin conjugate.

A pharmaceutical composition comprising a chlorotoxin conjugate can be formulated according to known methods to prepare pharmaceutically useful compositions, whereby the conjugate is combined with a pharmaceutically acceptable carrier. A composition is said to be a “pharmaceutically acceptable carrier” if its administration can be tolerated by a recipient patient. Sterile phosphate-buffered saline is one example of a pharmaceutically acceptable carrier. Other suitable carriers are well-known to those in the art. See, for example, Gennaro (ed.), Remington's Pharmaceutical Sciences, 19th Edition (Mack Publishing Company 1995).

A pharmaceutical composition comprising a chlorotoxin conjugate can be furnished in liquid form, in an aerosol, or in solid form. Liquid forms, are illustrated by injectable solutions, aerosols, droplets, topological solutions and oral suspensions. Exemplary solid forms include capsules, tablets, and controlled-release forms. The latter form is illustrated by miniosmotic pumps and implants (Bremer et al., Pharm. Biotechnol. 10:239 (1997); Ranade, “Implants in Drug Delivery,” in Drug Delivery Systems, Ranade and Hollinger (eds.), pages 95-123 (CRC Press 1995); Bremer et al., “Protein Delivery with Infusion Pumps,” in Protein Delivery: Physical Systems, Sanders and Hendren (eds.), pages 239-254 (Plenum Press 1997); Yewey et al., “Delivery of Proteins from a Controlled Release Injectable Implant,” in Protein Delivery Physical Systems, Sanders and Hendren (eds.), pages 93-117 (Plenum Press 1997)). Other solid forms include creams, pastes, other topological applications, and the like.

Other dosage forms can be devised by those skilled in the art, as shown, for example, by Ansel and Popovich, Pharmaceutical Dosage Forms and Drug Delivery Systems, 5.sup.th Edition (Lea & Febiger 1990), Gennaro (ed.), Remington's Pharmaceutical Sciences, 19.sup.th Edition (Mack Publishing Company 1995), and by Ranade and Hollinger, Drug Delivery Systems (CRC Press 1996).

As an illustration, pharmaceutical compositions may be supplied as a kit comprising a container that comprises a chlorotoxin conjugate. Therapeutic conjugates can be provided in the form of an injectable solution for single or multiple doses, or as a sterile powder that will be reconstituted before injection. Alternatively, such a kit can include a dry-powder disperser, liquid aerosol generator, or nebulizer for administration of a therapeutic conjugate. Such a kit may further comprise written information on indications and usage of the pharmaceutical composition.

Various references, including patent applications, patents, and scientific publications, are cited herein, the disclosures of each of which is incorporated herein by reference in its entirety.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

All features discussed in connection with any aspect or embodiment herein can be readily adapted for use in other aspects and embodiments herein. The use of different terms or reference numerals for similar features in different embodiments does not necessarily imply differences other than those expressly set forth. Accordingly, the present invention is intended to be described solely by reference to the appended claims, and not limited to the embodiments disclosed herein.

Unless otherwise specified, the presently described methods and processes can be performed in any order. For example, a method describing steps (a), (b), and (c) can be performed with step (a) first, followed by step (b), and then step (c). Or, the method can be performed in a different order such as, for example, with step (b) first followed by step (c) and then step (a). Furthermore, those steps can be performed simultaneously or separately unless otherwise specified with particularity.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Where a range of values is provided, it is understood that each intervening value, to the tenth of the unit of the lower limit unless the context clearly dictates otherwise, between the upper and lower limit of that range, and any other stated or intervening value in that stated range, is encompassed within the disclosure provided herein. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the disclosure provided herein.

All features discussed in connection with an aspect or embodiment herein can be readily adapted for use in other aspects and embodiments herein. The use of different terms or reference numerals for similar features in different embodiments does not necessarily imply differences other than those expressly set forth. Accordingly, the present invention is intended to be described solely by reference to the appended claims, and not limited to the embodiments disclosed herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

The invention is further illustrated by the following non-limiting examples.

### Example 1

### Intraoperative Imaging of Canine Tumor and Peritumoral Skin

This example describes the tumor- and peritumor-binding specificity of a modified chlorotoxin peptide (having K15 and K23 of native chlorotoxin replaced with arginine) conjugated to a cyanine dye and the ratio between tumor/peritumor and background binding by the tumor conjugate.

Dogs with naturally occurring solid tumors were given a chlorotoxin conjugate intraveously before surgery. An intraoperative near-infrared (NIR) imaging system enabled gross imaging of canine tumors in situ and immediately ex vivo, as well as assessments of tumor bed and gross tumor to background ratios.

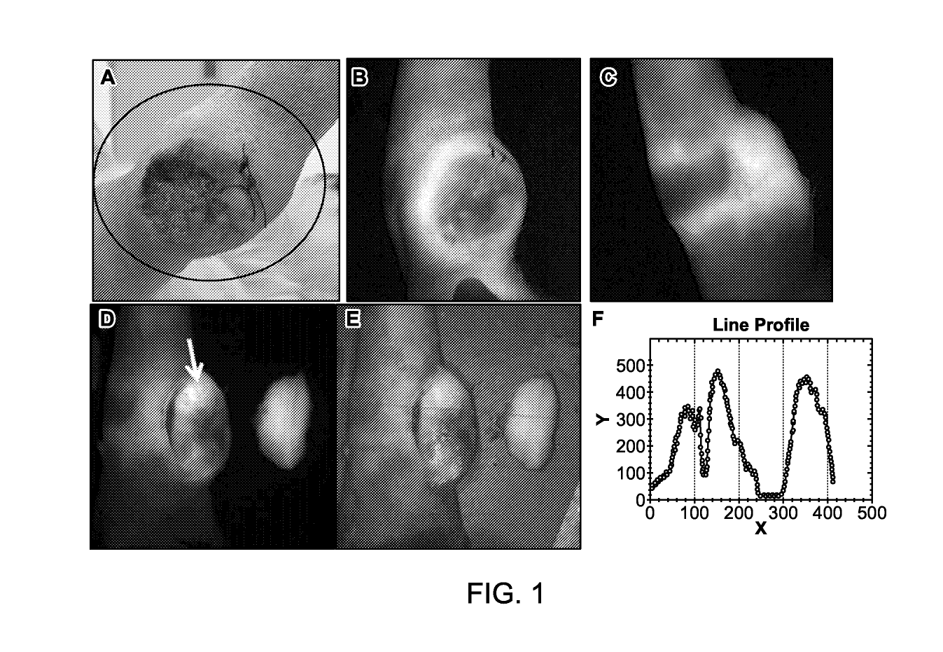

Patient 23 had a cutaneous squamous cell carcinoma of the tail. The lesion had penetrated the skin, which was grossly swollen and ulcerated (FIG. 1A). The lesion was covered by a serocellular crust. Preoperative fluorescence imaging showed very little fluorescence penetrating the serocellular crust, while the peritumoral skin showed relatively bright staining (FIG. 1B). Two “fingers” of fluorescence were noted, which extended to the opposite side of the tail (FIG. 1C).

Following removal of the tail, tissues were imaged and sections were removed for further imaging. A section of central tumor (FIG. 1D and FIG. 1E, at right) showed relatively intense fluorescence. The remaining central tumor, viewed from the side rather than through the serocellular crust, showed fluorescence intensity similar to that of the central tumor. Although the peritumoral skin was less intense than the tumor itself (FIG. 1F), it was about 3-fold more intense than uninvolved skin, suggesting that chlorotoxin conjugates have affinity for peritumoral tissue. Samples of skin from the fluorescent areas on the opposite side of the tumor were submitted for histopathology, and they did not contain tumor.

### Example 2

### Chlorotoxin Conjugate Labeling of Dermis Surrounding Cutaneous Squamous Cell Carcinoma

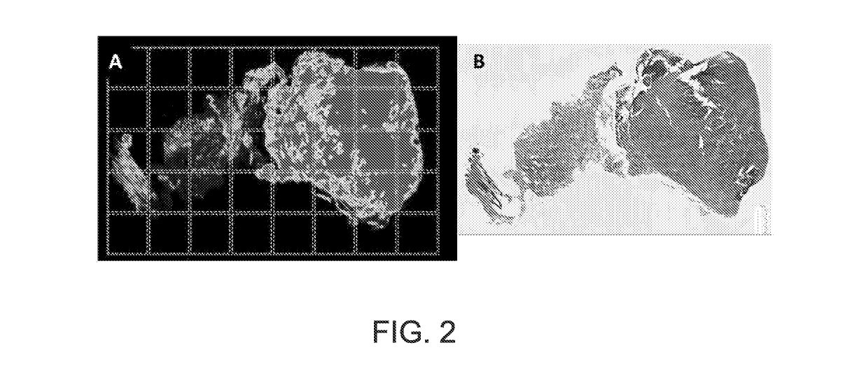

This example describes the evaluation of canine tumor and tumor microenvironment and adjacent tissues at the cellular level in order to assess sensitivity and specificity of a chlorotoxin conjugate for tumor microenvironment. For this analysis, two cutaneous squamous cell carcinomas, three mammary cancers, and three subcutaneous soft-tissue sarcomas were included. Tissues were sectioned on a cryostat, and 30 micron sections were imaged on the Odyssey scanner. These sections or serial sections were stained with H&E and read by an expert histopathologist who was blinded to the fluorescence data. A grid was overlaid on the fluorescence image (FIG. 2), and total fluorescence in each grid square was measured using Image Studio (Li-Cor) software provided with the Odyssey scanner. Overlay of the fluorescence image with the scored H&E image enabled calling of tumor vs. non-tumor for each grid square.

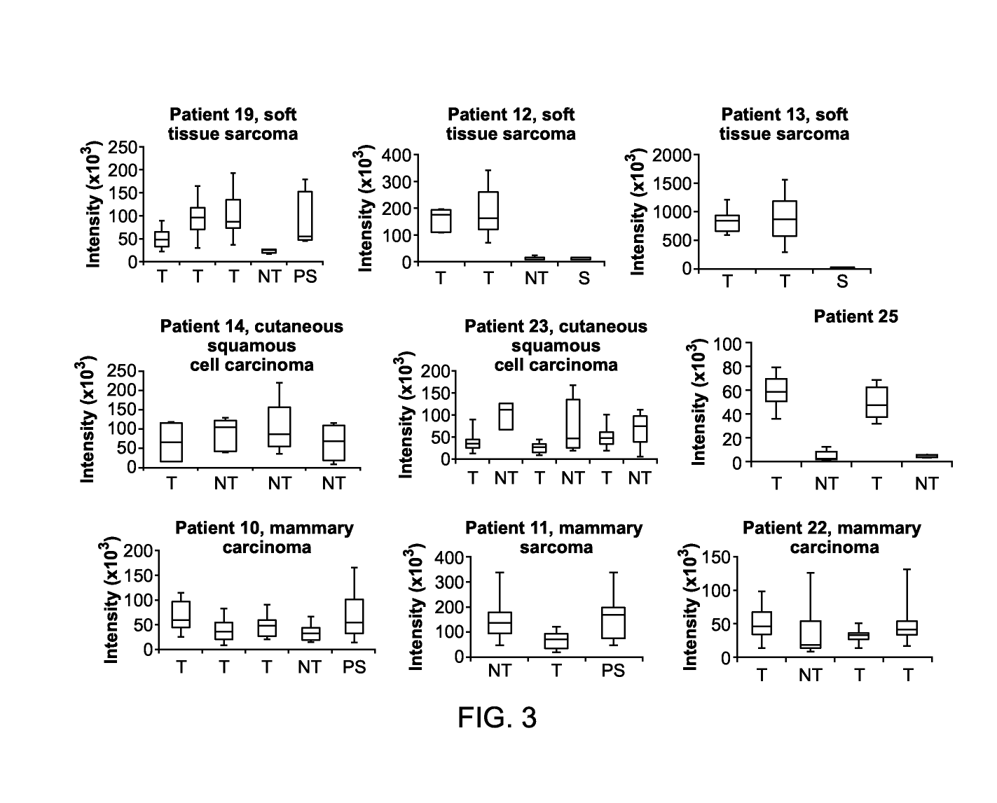

For each tumor, grid analysis was done on sections from different areas of tumor and adjacent non-tumor tissue, as well as samples of uninvolved tissue when available. The data were grouped by individual section and plotted for each patient. The results are shown in FIG. 3.

The cutaneous squamous cell carcinomas had fluorescence signal coming both from the tumor and from the underlying dermis. In most cases, the signal was brighter in the underlying dermis, leading to the “inverted” tumor vs. normal intensity. Although specificity in these tumors is low, truly uninvolved skin seen during intraoperative imaging in patient 23 was not fluorescent. It is possible that the tumors are secreting cytokines or growth factors that modify the microenvironment in the surrounding skin. Given the propensity of squamous cell carcinomas to spread locally, it is possible that modifications of the local microenvironment may precede invasion. Detailed study of this tumor type in humans may lead to a clinical application in which a chlorotoxin conjugate is used to highlight the region around the primary tumor that is modified by the tumor and at high risk of invasion.

Analysis of the mammary tumors shows that, like skin, mammary tissue adjacent to tumor takes up the chlorotoxin conjugate (a modified chlorotoxin peptide (having K15 and K23 of native chlorotoxin replaced with arginine) conjugated to a cyanine dye). Here too, the intraoperative imaging data enabled an assessment of tissues farther removed from the primary mass. These tissues did not have substantial background staining, suggesting that in mammary cancer a similar “penumbra” effect is present around tumor tissue. As discussed above, surgeons who treat human breast cancer are most concerned with residual cancer in the tumor bed following primary tumor removal. If a small lesion were missed, a penumbra of staining in the adjacent tissue would help make it detectable. Since breast cancer patients undergo second resections to remove missed tumor in 20-50% of cases, this is an area of intense interest.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of detecting a tumor microenvironment, peritumoral tissue, or cells therefrom, the method comprising the steps of:
a) administering a chlorotoxin conjugate to the tumor microenvironment, peritumoral tissue, or cells therefrom, wherein the chlorotoxin conjugate binds to the tumor microenvironment, peritumoral tissue, cells therefrom, or a combination thereof; and
b) imaging, visualizing, or analyzing the bound chlorotoxin conjugate.

2. The method of claim 1, further comprising removing the tumor microenvironment or cells therefrom bound by the chlorotoxin conjugate.

3. The method of claim 1, wherein the tumor microenvironment or cells therefrom are associated with tumors of skin or breast.

4. The method of claim 1, wherein the chlorotoxin conjugate is administered to an individual.

5. The method of claim 1, wherein the imaging, visualizing, or analyzing comprises visualizing the chlorotoxin conjugate optically.

6. The method of claim 1, wherein the imaging, visualizing, or analyzing comprises in vivo or ex vivo imaging, visualizing, or analyzing.

7. The method of claim 1, wherein the imaging, visualizing, or analyzing comprises optically imaging the tumor microenvironment.

8. The method of claim 1, wherein the chlorotoxin conjugate comprises a detectable label.

9. The method of claim 8, wherein the detectable label comprises a fluorescent moiety.

10. The method claim 9, wherein the fluorescent moiety comprises a near infrared fluorescent moiety.

11. The method of claim 8, wherein the detectable label comprises a cyanine dye.

12. The peptide of claim 8, wherein the detectable label is selected from the group consisting of Cy5.5, DyLight 750, indocyanine green (ICC) and IRdye 800.

13. The method of claim 8, wherein the detectable label comprises a radionuclide.

14. The method of claim 1, wherein the detecting is performed during or related to surgery or resection.

15. The method of claim 1, wherein the chlorotoxin comprises a sequence having at least 85% sequence identity to the sequence of MCMPCFTTDHQMARXCDDCCGGXGRGXCYGPQCLCR, wherein X is selected from K, A and R (SEQ ID NO: 1).

16. The method of claim 1, wherein the imaging, visualizing, or analyzing the bound chlorotoxin conjugate is performed on a sample.


SEQUENCE LISTING (7 sequences)

SEQ ID NO: 1 - protein, 36 aa, Artificial Sequence
  Description of Artificial Sequence Synthetic polypeptide
MCMPCFTTDHQMARXCDDCCGGXGRGXCYGPQCLCR

SEQ ID NO: 2 - protein, 36 aa, Artificial Sequence
  Description of Artificial Sequence Synthetic polypeptide
MCMPCFTTDHQMARKCDDCCGGKGRGKCYGPQCLCR

SEQ ID NO: 3 - protein, 36 aa, Artificial Sequence
  Description of Artificial Sequence Synthetic polypeptide
MCMPCFTTDHQMARXCDDCCGGXGRGKCYGPQCLCR

SEQ ID NO: 4 - protein, 36 aa, Artificial Sequence
  Description of Artificial Sequence Synthetic polypeptide
MCMPCFTTDHQMARRCDDCCGGRGRGKCYGPQCLCR

SEQ ID NO: 5 - protein, 36 aa, Artificial Sequence
  Description of Artificial Sequence Synthetic polypeptide
MCMPCFTTDHQMARRCDDCCGGAGRGKCYGPQCLCR

SEQ ID NO: 6 - protein, 36 aa, Artificial Sequence
  Description of Artificial Sequence Synthetic polypeptide
MCMPCFTTDHQMARACDDCCGGRGRGKCYGPQCLCR

SEQ ID NO: 7 - protein, 36 aa, Artificial Sequence
  Description of Artificial Sequence Synthetic polypeptide
MCMPCFTTDHQMARACDDCCGGAGRGKCYGPQCLCR